(19) United States
(12) Patent Application Publication
(10) Pub. No.: US 2021/0138055 A1
(43) Pub. Date: May 13, 2021

(54) COMPOSITIONS, METHODS AND USES FOR MODULATING THE TUMOR MICROENVIRONMENT TO ENHANCE ANTITUMOR IMMUNITY

(71) Applicant: COLORADO STATE UNIVERSITY RESEARCH FOUNDATION, Fort Collins, CO (US)

(72) Inventors: Steven Dow, Littleton, CO (US); Renata Impastato, Fort Collins, CO (US)

(21) Appl. No.: 17/156,401

(22) Filed: Jan. 22, 2021

Related U.S. Application Data
(63) Continuation of application No. PCT/US2019/041471, filed on Jul. 11, 2019.
(60) Provisional application No. 62/712,865, filed on Jul. 31, 2018.

(51) Int. Cl. A61K 39/00; A61K 45/06; A61P 35/00; A61K 31/4178; A61K 31/138

(52) U.S. Cl. CPC A61K 39/001121; A61K 45/06; A61P 35/00; A61K 2039/552; A61K 31/138; A61K 2039/55555; A61K 31/4178

(57) ABSTRACT

Embodiments herein report compositions, methods, and uses for enhancing the immune system in a subject in need of such modulation/treatment. In certain embodiments, compositions and methods disclosed herein concern compositions that can include, but are not limited to, at least one agent capable of reducing the activation of, or blocking an angiotensin II receptor (ARB) and C-C chemokine receptor 2 (CCR2) and/or blocking a beta receptor (Beta Blocker, BRB), to induce and/or enhance immune response in subject. In certain embodiments, combination agents disclosed herein can enhance an immune response against cancer in a subject in need thereof. In other embodiments, combination therapies disclosed herein modify the tumor microenvironment (TME) to enhance effects of anti-cancer therapies as a combination therapy or used alone to modify the immune system of the subject to treat cancer in a subject.

### CROSS-REFERENCE TO RELATED APPLICATIONS

This International Application claims the benefit of U.S. Provisional Application No. 62/712,865, filed on Jul. 31, 2018, which is incorporated herein by reference in its entirety for all purposes.

### FIELD

Embodiments herein report compositions, methods, and uses for treating cancer or a condition associated with cancer in a subject in need of such treatment. Other embodiments concern inducing an immune response in a subject, or modifying a tumor microenvironment to increase anti-tumor immune responses by the subject. In certain embodiments, compositions and methods disclosed herein concern compositions that can include, but are not limited to, cancer stem cells, cancer stem cell derived antigens, and one or more agents that modify the tumor microenvironment (TME). In other embodiments, a TME modifying agent can include one or more agent provided as a pharmaceutical compositions that reduces or inhibits angiotensin II receptor activity (ARBs) and/or C-C chemokine receptor 2 (CCR2) activities alone or in combination with propranolol or a propranolol-like agent or other beta receptor antagonist (BRB) to induce and/or enhance an immune response in a subject. Other embodiments can include combining one or more agent that reduces or inhibits angiotensin II receptor and/or CCR2 activity and one or more agents that reduces, inhibits or blocks beta receptors to induce an enhanced immune response in a subject. Some compositions and methods disclosed herein can be used to treat cancer, a cancer-associated condition, reduce relapse or prolong remission, reduce or prevent metastasis, or treat other immune-compromising conditions

### BACKGROUND

Tumors generally are made up of a heterogeneous population of cells with a number of distinct properties. Many tumor cells express antigens that are recognized by the immune system, and this immune recognition can be increased by administering various types of cancer vaccines. However, even in cases where the vaccine may elicit effective T cell immune responses against tumors, the overall benefit of the vaccine can be minimal due in part because of tumor microenvironments (TMEs), which consists of stromal cells, endothelial cells, myeloid cells (including macrophages) and extracellular matrix, which are immune suppressive and inhibitory to T cell responses. Therefore, for effective vaccination or improved immune responses against cancer, these inhibitory effects of the TME must be minimized. Currently, there are few reliably effective methods of modifying the TME clinically, for example, in part because new effective agents have not been identified or developed, or because the TME is resistant to attempted modification. Until new agents or compositions are identified to modify the TME, it will continue to be difficult to employ cancer vaccines effectively as immunotherapy to treat cancer. Therefore, agents that modify the TME are in need.

### SUMMARY

Embodiments herein report compositions, methods, and uses for treating cancer or a condition related thereto. Other embodiments disclosed herein concern modifying a tumor microenvironment (TME) pharmacologically to induce an immune response in a subject in order to ameliorate a condition in the subject. In certain embodiments, compositions and methods disclosed herein include compositions that can include, but are not limited to, cancer stem cells, cancer stem cell derived antigens, small molecule(s), biologic(s) or agent(s), and one or more TME modifying agent that can include, but is not limited to, one or more agent that reduces or inhibits angiotensin II receptor activity (ARBs), and one or more agents that reduces, inhibits or blocks beta receptors (beta-adrenergic blockers, BRBs) that alone or in combination, can be used in an immunogenic composition to induce and/or enhance an immune response in the subject. In accordance with these embodiments, a treatment regimen of the instant invention can modulate an immune response, deplete tumor macrophages, and stimulate tumor T cell responses and infiltration into tumor tissues

Other embodiments disclosed herein can include combining one or more agent that reduces or inhibits angiotensin II receptor activity (ARBs) and CCR2 activity to for example, deplete tumor macrophages to modify the TME, along with propranolol or other BRB to also deplete tumor macrophages and stimulate T cell responses. In other embodiment, compositions disclosed herein can modify a TME and/or reduce, inhibit or block beta receptor stimulation (beta-adrenergic blockers, BRBs) to induce an enhanced immune response in the subject (e.g. induction of effector T-cells specific for tumor antigens). In accordance with these embodiments, combinations of ARBs with BRBs can be used to treat a condition or prevent onset of a condition in a subject by inducing an enhanced immune response in the subject. In other embodiments, compositions disclosed herein can he used to target cancer stem cells (CSCs) or other tumor antigens with cancer vaccines, for example, to improve patient survival, reduce or prevent cancer relapse and/or enhancing quality of life of a subject having cancer. In accordance with these embodiments, cancer targeting vaccines can be used in combinations with one or more agent that reduces or inhibits angiotensin II receptor activity (ARBs) or related chemokine receptor activity (e.g., CCR2 signaling). One or more agents that reduces, inhibits or blocks beta receptor stimulation (beta-adrenergic blockers, BRBs) or related receptor signaling; for example, to reduce tumor growth and/or metastasis.

In some embodiments, immunogenic compositions disclosed herein can include an antigen derived from a tumor. In other embodiments, compositions can include an antigen derived from a tumor or can include a tumor lysate, and further include a first agent of a family of agents that inhibits angiotensin II receptor (ARBs); and a second agent from a family of agents that inhibits or blocks beta receptors (BRBs). In accordance with these embodiments, the antigen derived from a tumor can include a lysate of cancer cells grown under specific serum-free and non-attached conditions in vitro and having elevated expression of one or more cancer stem cell-related markers. In other embodiments, cancer stem-cell related markers expressed by the in vitro grown tumor cells contemplated herein can include, but are not limited to CD90, CD117, Sca1, CD133, CD34, CD73, Nanog, Klf4, Oct3/4, and aldehyde dehydrogenase (ALDT) or other cancer stem cell affiliated marker, which can be elevated when compared to normal tumor cell populations from in vitro grown tumor cell lines grown under conditions of adherence and in fetal bovine serum, and extracts thereof. For example, extracts can include extracts from tumor cells grown under plastic adherence conditions in the presence of fetal bovine serum. In other embodiments, the antigen can include a lysate of tumor cells disclosed herein.

In accordance with these embodiments, a first agent that reduces or inhibits angiotensin II receptor activity (ARBs) and/or CCR2 receptor activity or signalling can include, but is not limited to, losartan, azilsartan, candesartan, eprosartan, irbesartan, olmesartan, telmisartan, talsartan, other ARBs or any combination thereof. In other embodiments, a second agent that inhibits the activity of or reduces binding to a beta receptor (BRBs) can include, but are not limited to, acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, ICI-118,551, SR 59230A, propranolol-like molecules or other BRBs or any combination thereof. In certain embodiments, the first agent can include losartan and/or telmisartan, and the second agent can include propranolol and/or carvedilol. In other embodiments, the first agent can include losartan and/or telmisartan, and the second agents include carvedilol and propranolol in order to enhance the immune response in a subject. In accordance with these embodiments, the composition can further include at least one immunogenic or vaccine adjuvant.

In other embodiments, compositions disclosed herein can include a composition for inducing an enhanced immune response, comprising: a first agent that inhibits the activity of an angiotensin II receptor and/or CCR2 receptor, and a second agent that inhibits the activity of a beta receptor. In accordance with these embodiments, the first agent that inhibits the activity of an angiotensin II receptor can include, but are not limited to, losartan, azilsartan, candesartan, eprosartan, irbesartan, olmesartan, telmisartan, talsartan, or any combination thereof. In certain embodiments, the second agent that inhibits the activity of a beta receptor can include, but are not limited to, acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, ICI-118,551, SR 59230A, other beta receptor blockers, propranolol-like molecules or any combination thereof. In certain embodiments, the first agent can include losartan and/or telmisartan, and the second agent can include at least one of carvedilol and/or propranolol. In certain embodiments, the combination compositions disclosed herein are capable of synergistic effects on TME by enhancing the immune response in a subject. In certain embodiments, the composition can further include at least one adjuvant in order to further enhance an immune response in a subject.

In yet other embodiments, compositions disclosed herein can be used for inducing an immune response by administering along with a cancer vaccine to achieve a synergistic effect for treating cancer in a subject or for increasing anti-tumor activity. In certain embodiments, these combination therapies can be used for reducing or inhibiting CCR2 signalling in order to reduce monocyte migration to tumor tissues or to lymph nodes in a subject. In some embodiments, prolonged reduction of monocyte migration can lead to the induction of macrophage depletion in a tumor or in lymph nodes tumor responding lymph nodes) of a subject. In other embodiments, combination therapies contemplated herein can have synergistic effects by modulating immune suppressive TME, depleting macrophages, inducing T cell infiltration into tumor tissues and/or enhancing anti-tumor activity compared to using cancer vaccines, ARBs and beta receptor blockers (BRB) alone. In accordance with these embodiments, compositions disclosed herein can be used to treat breast cancer, bone cancer, brain cancer (e.g. glioblastomas) or other cancers in a subject or for inducing an enhanced immune response. In certain embodiments, compositions or combination treatments disclosed herein can be used to modify TME in combination with inducing T-cell targeted immunotherapies for improving tumor control, while avoiding harmful side effects such as drug toxicity.

In some embodiments, compositions of the instant disclosure can include a composition that includes any one of the compositions disclosed herein and a pharmaceutically acceptable carrier or excipient. Alternatively, compositions of the instant disclosure can include an immunogenic composition that includes an antigen, an ARB and a BRB disclosed herein, and a pharmaceutically acceptable carrier or excipient. The pharmaceutical compositions can be formulated for administration to the subject for delivery subcutaneously, intramuscularly, intradermally, intranasally, orally, topically, transdermally, parenterally, gastrointestinally, transbronchially or transalveolarly or mucosally.

It is contemplated herein that compositions and combination therapies disclosed can include other agents that either modulates macrophage influx or presence in a tumor and/or agents that target cancer-promoting macrophages (e.g. M2 phenotype macrophages) and/or agents that promote immune-boosting macrophages (e.g. M1 phenotype macrophages). In certain embodiments, additional agents contemplated herein can be cytokines (eg, interferons or interleukins) or ligands for Toll-like receptors (eg, TLR3 or TLR9 agonists), or ligands for NOD-like receptors (eg, muramyl tripeptide), or other agents that activate macrophages to reduce their immune suppressive properties found in cancers, and to elicit anti-tumor activity.

Embodiments herein concern compositions, methods and uses for inducing an immune response against cancers by altering the TME to become less immune suppressive. In some embodiments, compositions and methods disclosed herein can be combined with other cancer treatments, including, but not limited to tumor vaccines targeting tumor antigens, checkpoint molecule targeted immunotherapeutics, with cytotoxic chemotherapy, with targeted cancer drugs (e.g., Sunitinib), or with radiation therapy to improve the overall effectiveness of cancer therapies.

Yet other embodiments relate to compositions, methods and uses for treating cancer or a condition associated with cancer. In accordance with these embodiments, methods disclosed herein can include methods for treating cancer or a condition associated with cancer in a subject by administering a therapeutically effective amount of a pharmaceutical composition disclosed herein. In accordance with these embodiments, the pharmaceutical compositions disclosed herein can be used to block monocyte migration or other immune response, reduce or inhibit tumor angiogenesis, reduce or inhibit metastasis of a tumor and/or targeting reduction or elimination of CSCs in a tumor in order to reduce or prevent relapse. In certain embodiments, pharmaceutical compositions disclosed herein are used to inhibit and/or assist in reducing growth, expansion or metastasis of cancer in the subject, for example, any type of cancer such as melanoma, breast cancer, and glioblastoma.

In certain embodiments, compositions and methods disclosed herein can further include, one or more combination therapies including, but not limited to, administering at least one additional T-cell targeted, myeloid-cell targeted, adoptive cell transfer, innate immune activating molecule, or metabolic pathway inhibitor immunotherapy to the subject. In accordance with these embodiments, the at least one additional T-cell targeted immunotherapy comprises one or more checkpoint molecule targeted therapy or adoptive cell therapy.

### BRIEF DESCRIPTION OF THE FIGURES

The following drawings form part of the present specification and are included to further demonstrate certain embodiments. Some embodiments may be better understood by reference to one or more of these drawings alone or in combination with the detailed description of specific embodiments presented.

FIG. 1 represents an exemplary graph illustrating an additive or synergistic anti-tumor effect of a cancer vaccine (Vax), with or without an exemplary ARB (e.g. losartan) or when combined as an exemplary ARB plus an exemplary BRB (e.g. losartan plus propranolol), on the growth of an exemplary tumor of certain embodiments disclosed herein

FIG. 2 represents an exemplary graph illustrating an additive anti-tumor effect of an exemplary ARB (e.g. losartan) or an exemplary ARB (e.g. propranolol) or an exemplary ARB plus an exemplary BRB (e.g. losartan plus propranolol), on the growth of an exemplary tumor of certain embodiments disclosed herein.

FIG. 3 represents an exemplary graphs illustrating the T cell infiltration stimulatory effects of the combination of an exemplary ARB (e.g. losartan), an exemplary BRB (e.g. propranolol), an exemplary BRB (e.g. carvedilol), an exemplary ARB plus an exemplary BRB (e.g. losartan plus propranolol), or an exemplary ARB plus an exemplary BRB (e.g. losartan plus carvedilol) (10 mg/kg once daily, i.p.) compared to an untreated control, on the infiltration of CD45⁻ immune cells (e.g., T cells, CD4+ T cells, CD8+ T cells, right column) into tumor tissues of certain embodiments disclosed herein.

FIG. 4 represents an exemplary histogram plot illustrating inhibitory effects of an exemplary ARB (e.g. losartan), an exemplary BRB (e.g. propranolol) compared to a negative and positive control, on monocyte migration of certain embodiments disclosed herein.

FIG. 5 represents an exemplary graph illustrating percent survival of test animals having an established tumor (e.g. brain tumor, GL261 model) and effects of an exemplary ARB (e.g. losartan) plus an exemplary BRB (e.g. propranolol) with and without a tumor lysate vaccine compared to an untreated control animals of certain embodiments disclosed herein.

FIG. 6 represents an exemplary graph illustrating effects of an exemplary ARB (e.g. losartan) or an exemplary ARB (e.g. propranolol) or an exemplary ARB plus an exemplary BRB (e.g. losartan plus propranolol) on increased CDS T cell infiltration into tumors compared to an untreated control animals of certain embodiments disclosed herein.

FIG. 7 represents an exemplary graph illustrating effects of an exemplary ARB (e.g. losartan) or an exemplary ARB (e.g. propranolol) or an exemplary ARB plus an exemplary BRB (e.g. losartan plus propranolol) on increased CD4 T cell infiltration into tumors compared to an untreated control animals of certain embodiments disclosed herein.

FIG. 8 represents exemplary images illustrating tumor regression before and after treatment with an exemplary ARB (e.g. losartan) plus an exemplary BRB (e.g. propranolol) in combination with a tumor vaccine treatment (e.g. allogeneic cancer vaccine) in an animal model of certain embodiments disclosed herein.

FIG. 9 represents an exemplary graph illustrating the effect of conventional tumor vaccine (Tumor Vax, square) and an enhanced vaccine (Spheroid Vax: VAX, triangle) on the growth or expansion of cutaneous tumors in mice compared to a control group of animals (closed circle) after the indicted time periods of certain exemplary embodiments herein.

FIG. 10 represents an exemplary graph illustrating an effect of conventional tumor vaccine (Tumor Vax, squares) and an enhanced immune vaccine (Spheroid Vax: triangles) on the release of interferon gamma (IFN-γ) from spleen cells of vaccinated mice with tumors or control mice with tumors (e.g. CT26 tumor model, colon carcinoma) after vaccination with either enhanced immune or conventional tumor cell lysates of certain exemplary embodiments herein. (Note, cytokine markers can be measured as a T cell response indicator)

FIG. 11A represents (A) an exemplary plot illustrating an immune stimulatory effect of cancer cell antigens derived from cancer cells (e.g. 4T1 mouse mammary cancer cells) grown in tissue culture under conventional conditions (plastic) or under spheroid conditions (spheroid, which increases tumor immunogenicity and enriches for cancer stem cells) on levels of interferon gamma (IFN-γ) produced by mouse spleen cells (from C57B1/6 mice) of certain embodiments disclosed herein. (Of note: level of IFN-γ produced is a reflection of the overall inherent immunogenicity of the vaccine antigens and NK cell activity)

FIG. 11B represents (A) an exemplary plot illustrating and comparing an immune stimulatory effect of cancer cell antigens derived from cancer cells (e.g. 4T1 mouse mammary cancer cells) grown in tissue culture under conventional conditions (plastic) or under spheroid conditions (spheroid, which increases tumor immunogenicity and enriches for cancer stem cells) on levels of interferon gamma (IFN-γ) produced by mouse spleen cells (from CD-1 mice) of certain embodiments disclosed herein. (Of note: level of IFN-γ produced is a reflection of the overall inherent immunogenicity of the vaccine antigens and NK cell activity).

### DEFINITIONS

As used herein, the term “about” refers to a range of values plus or minus 10 percent, e.g., about 1.0 encompasses values from 0.9 to 1.1; about 10% encompasses values from 9.9% to 10.1%.

As used herein, the singular form “a”, “an”, and “the” includes plural references unless indicated otherwise.

The term, “treating” as used herein can include modulating a condition, for example by administering to a human or an animal subject at least one dose of a compound, agent or composition, treating includes lessening the likelihood such as onset and/or severity of at least one condition, as well as, limiting the length of or severity of the condition, treating can or cannot result in a cure of a condition or disease.

As used herein, the term “modulating” means changing, and includes positive modulating, such as “increasing,” “enhancing,” “inducing” or “stimulating,” as well as negative modulating such as “decreasing,” “inhibiting” or “reducing,” typically in a statistically significant or a physiologically significant amount as compared to a control.

As used herein, “inhibition” or “inhibitory activity” each encompass whole or partial reduction of activity or effect of an enzyme or all and/or part of a pathway that includes an enzyme that is effected either directly or indirectly by the inhibitor or a pathway that is effected either directly or indirectly by the activity of the enzyme which is effected either directly or indirectly by the inhibitor.

As used herein, unless explicitly stated otherwise or clearly implied the terms “therapeutically effective dose,” “therapeutically effective amounts,” and the like, refer to a portion or amount of a compound, composition or agent that has a net positive effect on health and well-being of a human or other animal subject. Therapeutic effects can include an improvement in longevity, quality of life and the like these effects also can also include a reduced susceptibility to developing disease or deteriorating health or well-being. The effects can be immediate realized after a single dose and/or treatment or they can be cumulative realized after a series of doses and/or treatments, A “therapeutically effective amount” in general means the amount that, when administered to a subject or animal for treating a disease, is sufficient to affect the desired degree of treatment for the disease.

Pharmaceutical formulations of the present invention suitable for oral administration can be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The formulation can also be a bolus, electuary or paste.

Pharmaceutical formulations of the present invention suitable for parenteral administration can include aqueous and non-aqueous sterile injection solutions, and can also include an antioxidant, buffer, a bacteriostat and a solution which renders the composition isotonic with the blood of the recipient, and aqueous and non-aqueous sterile suspensions which can contain, for example, a suspending agent and a thickening agent. The formulations can be presented in a single unit-dose or multi-dose containers, and can be stored in a lyophilized condition requiring the addition of a sterile liquid carrier prior to use.

Pharmaceutically acceptable carrier or excipient: Pharmaceutically acceptable carrier or excipient, unless stated or implied otherwise, can be used herein to describe any ingredient other than the active component(s) that can be included in a formulation. The choice of carrier will to a large extent depend on factors such as the particular mode of administration, the effect of the carrier on solubility and stability, and the nature of the dosage form.

A tablet can be made by compressing or moulding the active ingredient with the pharmaceutically acceptable carrier. Compressed tablets can be prepared by compressing in a suitable machine the active ingredient in a free-flowing form, such as a powder or granules, in admixture with, for example, a binding agent, an inert diluent, a lubricating agent, a disintegrating and/or a surface active agent. Moulded tablets can be prepared by moulding in a suitable machine a mixture of the powdered active ingredient moistened with an inert liquid diluent. The tablets can optionally be coated or scored and can be formulated so as to provide slow or controlled release of the active ingredient. In one example, one or more ARBs and one or more BRBs can be combined into a single tablet, pill, capsule or the like.

As used herein, “conditions” and/or “diseases” include, but are not limited to, autoimmunity, neurodegenerative diseases, cancers such as brain cancer, breast cancer, endocrine cancer, gastrointestinal cancer, gynecologic cancer, prostate cancer, head and neck cancer, hematologic cancer, lung cancer, renal cell carcinoma, skin cancer, urologic cancer, rare cancers, and/or clinical conditions associated with or induced by modulation of a gene. Exemplary clinical conditions that can be treated with methods disclosed herein include diseases as well as other clinical conditions that are caused by modulation of a gene. Modulation of a gene can include, but is not limited to, alteration of gene expression by an agent, stress, and/or environmental factors, Alteration of gene expression can include, but is not limited to, underexpression and/or overexpression of a gene.

As used herein, “tumor,” “cancer” and “cancerous” can refer to or describe the physiological condition in mammals that is typically characterized by unregulated cell growth. Included in this definition are benign and malignant tumors or cancers as well as dormant tumors or micrometastases. Examples of tumors or cancers include, but are not limited to, carcinoma, lymphoma, blastoma, sarcoma, and leukemia. Other examples of cancer include, but are not limited to, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and cancer-related conditions, for example, post-transplant lymphoproliferative disorder (PTLD), as well as abnormal vascular proliferation associated with phakomatoses, edema (such as that associated with brain tumors), and Meigs' syndrome.

An “immunogenic composition” can be used to refer to a composition that induces an immune response in a subject when introduced to the subject, for example “a vaccine.”

As used herein, “Angiotensin II Receptor Blockers (ARBs)” can be a group of pharmaceutical drugs that can modulate the renin-angiotensin-aldosterone system by selectively inhibiting the effects of angiotensin II (Ang II), which is a peptide hormone that plays an important role in the pathophysiology of hypertension. Exemplary ARBs can include, but are not limited to, losartan, azilsartan, candesartan, eprosartan, irbesartan, olmesartan, telmisartan, and talsartan.

As used herein, “Beta Blockers” or “Beta Receptor Blockers” can be a class of antagonists that can block the receptor sites for the endogenous catecholamines epinephrine (adrenaline) and norepinephrine (noradrenaline) on adrenergic beta receptors in the sympathetic nervous system. Some block activation of all types of β-adrenergic receptors and others are selective for one of the three known types of beta receptors, designated β₁, β₂, and β₃; receptors. Exemplary beta blockers can include, but are not limited to, acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, propranolol-like agents with TME-modifying activity, sotalol, timolol, IC1-118,551, and SR 59230A.

As used herein, the term “adjuvant” can have its conventional meaning, e.g., the ability to enhance an immune response in general or to a particular antigen. Such ability can be manifested by a significant increase in immune-mediated protection. An enhancement of humoral immunity can be manifested by a significant increase (usually about >10%) in the titer of antibody raised to the antigen. Similarly, enhancement of cellular immunity can be typically manifested by a significant increase (e.g. about 10% or more) in the number of CD8⁻ or CD4⁻ cells

In certain embodiments, a vaccine adjuvant can be any adjuvant known or used by one of skill in the relevant art, including but not limited to: CLDC (cationic liposome DNA complexes) adjuvants, mineral salts, such as aluminium salts and calcium salts, including hydroxides (e.g., oxyhydroxides), phosphates (e.g., hydroxyphosphates, orthophosphates) and sulphates, etc.; oil-in-water emulsions, such as squalene-water emulsions, including MF59 (5% Squalene, 0.5% Tween 80, and 0.5% Span 85, formulated into submicron particles using a microfluidizer); complete Freund's adjuvant (CFA) and incomplete Freund's adjuvant (IFA); saponin formulations, such as QS21 and ISCOMs; virosomes and virus-like particles (VLPs); bacterial or microbial derivatives, such as non-toxic derivatives of enterobacterial lipopolysaccharide (LPS), Lipid A derivatives; immunostimulatory oligonucleotides, such as IC-31 (deoxynucleotide comprising 2-mer sequence 5′-(IC)₁₃-3′ and polycationic polymer polypeptide comprising 11-mer amino acid sequence KLKLLLLLKLK) and ADP-ribosylating toxins and detoxified derivatives thereof; human immunomodulators, including cytokines, such as interleukins (e.g., IL-1, IL-2, IL-4, IL-5, IL-6, IL-7, IL-12, interferons interferon-gamma), macrophage colony stimulating factor, and tumor necrosis factor; bioadhesives and mucoadhesives, such as chitosan and derivatives thereof, esterified hyaluronic acid microspheres or mucoadhesives, such as cross-linked derivatives of poly(acrylic acid), polyvinyl alcohol, polyvinyl pyrollidone, polysaccharides and carboxymethylcellulose; microparticles (e.g., a particle of about 100 nm to about 150 um in diameter) formed from materials that are biodegradable and non-toxic (e.g., a poly(alpha-hydroxy acid), a polyhydroxybutyric acid, a polyorthoester, a polyanhydride, a polycaprolactone, etc.); liposomes; polyoxyethylene ethers and polyoxyethylene esters; PCPP formulations; muramyl polypeptides, including N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE); and imidazoquinolone compounds, including Imiquamod and its homologues (e.g. “Resiquimod 3M”). Illustrative adjuvants suitable for use include, but are not limited, to cationic lipid DNA complexes (CLDC), cationic lipid DNA pI:C complexes (CLDPC), CpG-oligonucleotides, poly I:C, LPS, alpha-galactosylceramide, and the like.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some cases, well-known methods or components have not been included in the description.

In accordance with embodiments, there may be employed conventional molecular biology, protein chemistry, microbiology, cell biology and recombinant DNA techniques within the skill or the art. Such techniques are explained fully in the literature.

In certain embodiments, compositions and methods disclosed herein can be used to identify known agents having other medical applications that are capable of modifying TME for treating immune disorders as well as cancer. In accordance with these embodiments, libraries of existing agents can be screened to identify those that may have “off-target” effects on the immune system. Agents screened for off-target effects can include agents capable of blocking migration of white blood cells known as monocytes, which are the precursor cells to tumor macrophages, one of the major immune suppressive cell populations in the TME. Using these screening methods, angiotensin receptor antagonists (ARBs) agents that normally function by blocking signaling by the angiotensin II receptor, were identified as being able to block monocyte migration. In certain embodiments, ARBs can be used to inhibit monocyte migration. This effect may be due in part to inhibiting activation of the C-C chemokine receptor 2 (CCR2), which is the primary receptor used by monocytes for migration to sites of cancer. In certain embodiments, the ARB is losartan.

In other embodiments, propranolol or propranolol-like agents or other BRBs, in a second class of agents known as beta receptor blockers (BRBs) are capable of blocking monocyte migration, as well as capable of increasing T cell infiltration into tumors. In certain embodiments, when an ARB (e.g. losartan) and a BRB (e.g. propranolol or propranolol-like agent) are co-administered, they exert synergistic immune activity and modification of the TME by for example, depleting tumor macrophages. Therefore, combination compositions of ARBs and BRBs are novel compositions of known agents for use in novel methods for pharmacologically modifying the TME and/or enhancing an immune response, which is distinct from their original intended and currently marketed uses as agents to modify blood pressure or heart rate. These agents are currently marketed individually providing an advantage to identifying unknown uses of these known compounds as well as identifying novel combinations not currently thought to have function& uses together for at least additive and as demonstrated, synergistic effects of enhancing immune responses in a subject. Alone or in combination, ARBs and/or BRBs can significantly slow tumor growth and/or migration in a subject.

In other embodiments, ARB and BRB combinations can be co-administered with a cancer vaccine to increase the overall effectiveness of the cancer vaccine, with reduced side-effects of known for other agents. In certain embodiments, the combination therapy can be used to modify the TME in order to improve overall efficacy of other cancer immunotherapeutics.

Embodiments herein report compositions, methods, uses for modulating/treating cancer by reducing tumor size, enhancing immunity (e.g. anti-tumor immunity) and/or reducing or eliminating expansion or metastasis of a tumor or spread of the cancer or treating a condition associated with cancer in a subject in need of such modulation/treatment.

In certain embodiments, compositions and methods disclosed herein concern compositions that can include, but are not limited to, a first agent that inhibits or blocks activity and/or binding of an angiotensin II receptor (ARBs); and a second agent that inhibits or blocks beta receptor (BRBs) binding or activity, and to vaccines against cancer antigens, including cancer stem cell antigens. In accordance with these embodiments, the cancer antigens can include antigens prepared by lysing tumor cells grown as cell lines in vitro. In particular, tumor cells grown under conditions (e.g., non-adherent cultures in serum-free medium) capable of increasing overall immunogenicity of the antigens and upregulate expression of cancer stem cell antigens, are one source of tumor antigens for vaccines that can be combined with compositions disclosed herein.

In other embodiments, tumor antigen compositions can be produced by subjecting tumor cell lines to 1, or 2, or 3, or 4, or 5 or more freeze-thaw cycles and creating tumor cell lysates. In certain embodiments, these tumor cell lysates whether subjected to freeze-thaw or not, can be partially purified to generate tumor cell lysates from these conditioned cells. In other embodiments, these populations can be sonicated to generate a more homogenous composition of tumor cell lysates with enhanced immunogenicity. In some embodiments, the antigens of compositions contemplated herein can include a lysate of tumor cells either partially or wholly purified. In other embodiments, tumor cell lysates, when admixed with a pre-selected vaccine adjuvant, can act to induce an immune response (e.g. T cell and antibody responses) against antigens highly expressed (surface or intracellular expression) by cancer cells.

In other embodiments, antigens of use in compositions disclosed herein can include a lysate of tumor cells. In certain embodiments, tumor cells disclosed herein can include at least one cell from one or more cancers comprising squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulvar cancer, thyroid cancer, hepatic carcinoma, various types of head and neck cancer, glioblastoma, B-cell lymphoma, low grade/follicular non-Hodgkin's lymphoma (NHL), small lymphocytic (SL) NHL, intermediate grade/follicular NHL; intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, chronic lymphocytic leukemia (CLL), acute lymphoblastic leukemia (ALL), Hairy cell leukemia, and chronic myeloblastic leukemia.

In some embodiments, compositions and methods disclosed herein can include administering combination therapies of ARBs/BRBs and/or a cancer cell lysate or other tumor vaccines and further include other forms of cancer immunotherapy, including, but not limited to, checkpoint targeted antibodies (e.g., antibodies targeting PD-a, PD-L1, CTLA-4, OX40, and other checkpoint molecules), cytokines, biological response modifiers, and inhibitors of immune suppressive biochemical pathways (e.g., indoleamine deoxygenase pathway, arginase pathways, glutamine synthesis, tryptophan synthesis). Other immune modulators include cellular therapy with activated T cells or NK cells, or chimeric antigen receptor T cells (CAR-T cells).

In other embodiments, compositions and methods disclosed herein can include administering combination therapies of ARBs/BRBs and/or a cancer antigen vaccine and further include one or more of the aforementioned immune modulators for cancer Angiotensin II Receptor Blockers (ARBs).

Angiotensin II receptor blockers (ARBs) are agents that are known to block the action of angiotensin II by preventing angiotensin II from binding to angiotensin II receptors on the muscles surrounding blood vessels. As a result, blood vessels enlarge (dilate) and blood pressure is reduced. Another activity of ARBs is the effect on other G-protein coupled receptors (GPCR) promiscuously, including the receptor CCR2. This “off target” activity of ARBs leads in part to immune modulation by blocking monocyte migration. One ARB, Losartan, was the first ARB to be developed and approved by the FDA and has served as a platform for the development of other ARBs, such as, Azilsartan, Candesartan, Eprosartan, Irbesartan, Olmesartan, Telmisartan, and Valsartan and other ARBs. In certain embodiments, compositions and methods disclosed herein include administering ARBs and BRBs alone or in combination with cancer antigens (e.g. cancer antigen vaccines) for administration to a subject having cancer or having a condition requiring immunomodulation or enhancement. As disclosed herein, in certain embodiments, ARBs can be used in compositions alone or in compositions containing BRBs of the instant invention to stimulate the production of T-cells, for example, CD4+ T-cells and CDS+ T-cells, to enhance an immune response against cancer or other immunological condition in a subject.

### Beta Receptor Blocker (BRBs)

Beta blockers, also referred to as β-blockers, include a class of agents that are often used to manage abnormal heart rhythms, and to protect the heart from a second heart attack (myocardial infarction) after a first heart attack (secondary prevention). Other uses for BRBs include treating high blood pressure (hypertension), although they are no longer the first choice for initial treatment of most patients. Beta blockers are competitive antagonists that block receptor interaction by endogenous catecholamines epinephrine (adrenaline) and norepinephrine (noradrenaline) on adrenergic beta receptors, of the sympathetic nervous system, which are known to mediate the fight-or-flight response. Other BRBs block activation of all types of β-adrenergic receptors and others are selective for one of the three known types of beta receptors, designated β1, β2 and β3 receptors. β1-adrenergic receptors are located mainly in the heart and in the kidneys. β2-adrenergic receptors are located mainly in the lungs, gastrointestinal tract, liver, uterus, vascular smooth muscle, and skeletal muscle. β3-adrenergic receptors are located in fat cells.

In addition, beta blockers interfere with the binding to the receptor of epinephrine and other stress hormones, and weaken the effects of stress hormones. In 1964, the first BRBs were synthesized, propranolol (e.g. Inderal™) and pronethalol. in addition to their effect on blood vessels and heart rhythm, it is also apparent that propranolol affects immune function, by blocking monocyte migration and stimulating I cell survival. As disclosed herein, in certain embodiments, BRBs can be part of a composition of use to stimulate the production of T-cells, for example, CD4+ T-cells and CDS+ T-cells to enhance an immune response against cancer or other condition in a subject. In certain embodiments, compositions disclosed herein can be introduced intravenously, orally or rectally. In other embodiments, compositions disclosed herein can be delivered for short, quick treatment or for a prolonged long-acting formulation.

In certain embodiments, at least a first agent that inhibits or blocks the activity or binding of an angiotensin II receptor (ARB) and/or the CCR2 receptor can include, but are not limited to, losartan, telmisartan, irbresartan azilsartan, candesartan, eprosartan, olmesartan, talsartan, or any combination thereof. In other embodiments, a second agent that inhibits or blocks beta receptor (BRBs) binding or activity can include, but are not limited to, acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, IC1-118,551, SR 59230A, or any combination thereof The first and second agent can be used in combination or alone or in combination with a cancer vaccine or other cancer immunotherapy. In one embodiment, at least one first agent can include losartan and at least one second agent includes propranolol and optionally, includes a cancer vaccine, prepared as tumor cell lysates or purified cancer antigens (proteins or peptides).

In certain embodiments, compositions disclosed herein can further include at least one adjuvant and/or co-stimulatory adjuvant. In accordance with these embodiments, adjuvants can include, but are not limited to, CLDC adjuvants, mineral salts, such as aluminium salts and calcium salts, including hydroxides (e.g., oxyhydroxides), phosphates (e.g., hydroxyphosphates, orthophosphates) and sulfates, etc.; oil-in-water emulsions, such as squalene-water emulsions, including MF59 (5% Squalene, 0.5% Tween 80, and 0.5% Span 85, formulated into submicron particles using a microfluidizer); complete Freund's adjuvant (CFA) and incomplete Freund's adjuvant (IFA); saponin formulations, such as QS21 and ISCOMs, virosomes and virus-like particles (VLPs) bacterial or microbial derivatives, such as non-toxic derivatives of enterobacterial lipopolysaccharide (LPS), Lipid A derivatives; immunostimulatory oligonucleotides, such as IC-31 (deoxynucleotide comprising 26-mer sequence 5′-(IC)13-3′ and polycationic polymer polypeptide comprising 11-mer amino acid sequence KLKLLLLLKLK) and ADP-ribosylating toxins and detoxified derivatives thereof; human immunomodulators, including cytokines, such as interleukins (e.g., IL-1, IL-2, IL-4, IL-5, IL-6, 1L-7, IL-12, interferons (e.g., interferon-gamma), macrophage colony stimulating factor, and tumor necrosis factor, bioadhesives and mucoadhesives, such as chitosan and derivatives thereof, esterified hyaluronic acid microspheres or mucoadhesives, such as cross-linked derivatives of poly(acrylic acid), polyvinyl alcohol, polyvinyl pyrollidone, polysaccharides and carboxymethylcellulose; microparticles (e.g., a particle of about 100 nm to about 150 um in diameter) formed from materials that are biodegradable and non-toxic (e.g., a poly(alpha-hydroxy acid), a polyhydroxybutyric acid, a polyorthoester, a polyanhydride, a polycaprolactone, etc.); liposomes; polyoxyethylene ethers and polyoxyethylene esters; PCPP formulations; muramyl polypeptides, including N-acetyl-murarnyl-L-threonyl-D-isoglutarnine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE); and imidazoquinolone compounds, including lmiquamod and its homologues (e.g. “Resiquimod 3M”) or other adjuvant. In one embodiment, an adjuvant of compositions disclosed herein can include cationic lipid DNA complexes (CLDC), cationic lipid DNA pI:C complexes (CLDPC), CpG-oligonucleotides, poly I:C, LPS, alpha-galactosylceramide, and the like. In other embodiments, the adjuvant can include a cationic liposome-DNA complex (CLDC) and/or a cationic liposome DNA-pIC complex (CLDPC).

In certain embodiments, antigen concentrations can be from about 1 μg to about 1000 μg of proteins, from about 10 μg to about 500 μg, of proteins, from about 20 μg to about 400 μg of proteins, from about 30 μg to about 300 μg of proteins, from about 40 μg to about 200 μg of proteins, from about 50 μg to about 100 μg of proteins, about 30 μg, about 40 μg, about 50 μg, about 60 μg, about 70 μg, about 80 μg, about 90 μg, about 100 μg of proteins, or any combination thereof. In other embodiments, the concentration of the first agent, the second agent, and/or the at least one adjuvant can be from about 0.001 g/L to about 1 g/L, about 0.01 g/L to about 1 g/L, about 0.05 g/L to about 1 g/L, about 0.1 g/L to about 1 g/L, about 0.25 g/L to about 1 g/L, about 0.1 g/L to about 0.5 g/L, about 0.1 g/L to about 0.25 g/L, or any combination thereof.

In other embodiments, concentrations of ARBs and/or BRBs, and/or adjuvant can be from about 0.001 g/L to about 1 g/L, about 0.01 g/L to about 1 g/L, about 0.05 g/L to about 1 g/L, about 0.1 g/L to about 1 g/L, about 0.25 g/L to about 1 g/L, about 0.1 g/L to about 0.5 g/L, about 0.1 g/L to about 0.25 g/L, or any combination thereof. In certain embodiments, the dose for ARB agents and/or BRB agents can be 2 times, 3 times, 4 times, 5 times, or 10 or 20 times more than normally prescribed.

In certain embodiments, dosage levels for ARB agents can be 2 times, 3 times, 4 times, 5 times or 10 or 20 times more compared to the normally prescribed ARES. Under standard uses for inhibiting or blocking binding of an angiotensin II receptor; for example, losartan normal dosing range is about 25 to about 150 mg/day. In certain embodiments, treating a pet (e.g. a dog) for cancer losartan or other ARB dose is 10-times higher than normal (e.g., 10 mg/kg BID for example compared to for example, 1 mg/kg BID for treating hypertension).

In some embodiments, compositions disclosed herein can include a composition that includes any one of the compositions or agents or combination of agents disclosed herein and a pharmaceutically acceptable carrier or excipient. Alternatively, compositions of the instant disclosure can include an immunogenic composition that comprises an antigen disclosed herein, and a pharmaceutically acceptable carrier or excipient. The pharmaceutical compositions can be formulated for administration to the subject for delivery subcutaneously, intramuscularly, intradermally, intravenously, intradermally, intranasally, orally, topically (including by ocular administration), transdermally, parenterally, gastrointestinally, transbronchially, mucosally, or transalveolarly.

In accordance with these embodiments, certain compositions disclosed herein can include an agent that inhibits the activity of an angiotensin II receptor, e.g., 10 mg/kg, at least about 20 mg, at least about 30 mg, at least about 40 mg, at least about 50 mg, at least about 100 mg, at least about 200 mg, at least about 300 mg, at least about 400 mg, at least about 500 mg, at least about 600 mg, at least about 700 mg, at least about 800 mg, including any range between these values, or less than about 400 mg, less than about 300 mg, less than about 200 mg, or less than about 100 mg, including any range between these values. In certain embodiments, an agent that inhibits the activity of an angiotensin II receptor is at a concentration sufficient to provide a dose of more than about 600 mg, more than about 750 mg, more than about 900 mg, more than about 1050 mg, or more than about 1200 mg, including any range in between these values, or any combinations thereof. In accordance with these embodiments, the agent that inhibits the activity of an angiotensin II receptor, can include, but are not limited to, at least one agent from losartan, azilsartan, candesartan, eprosartan, irbesartan, olmesartan, telmisartan, talsartan, or any combination thereof.

In certain embodiments, compositions disclosed herein can include an agent that inhibits or blocks activity and/or binding of an angiotensin II receptor (ARBs); at a concentration sufficient to provide a dose of at least about 0.5 mg/kg, at least about 0.75 mg/kg, at least about 1.0 mg/kg, at least about 1.25 mg/kg, at least about 1.5 mg/kg, at least about 1.75 mg/kg, or at least about 2.0 mg/kg of the first agent, including any range between about 0.5 mg/kg and about 1.75 mg/kg. In certain embodiments, compositions can include one or more ARBs at a concentration sufficient to provide a dose of more than about 1.75 mg/kg, at least about 2.0 mg/kg, at least about 5 mg/kg, at least about 7 mg/kg, at least about 10 mg/kg, at least about 12 mg/kg, at least about 15 mg/kg, at least about 17 mg/kg, at least about 20 mg/kg, at least about 22 mg/kg, at least about 25 mg/kg, at least about 27 mg/kg, or at least about 30 mg/kg, including any range in between about 1.75 mg/kg and about 30 mg/kg, wherein the ARB composition is at a concentration sufficient to provide a dose of at least about 0.1 to about 25 kg/mg: or about 0.5 to about 25 mg/kg; or about 1.0 to about 20 mg/kg or about 1.0 to about 15 mg/kg in a subject. or about 2.0 mg/kg to about 10 mg/kg or any combinations thereof in order to induce monocyte blockade in the subject. In certain embodiments, the compositions can include first agent at a concentration sufficient to provide a dose of more than about 30 mg/kg, e.g., at least about 35 mg/kg or at least about 40 mg/kg of Losartan, including any range in between about 30 mg/kg and about 40 mg/kg. Consistent with these embodiments, the ARB that inhibits the activity of an angiotensin II receptor can include, but are not limited to, at least one agent from losartan, azilsartan, candesartan, eprosartan, irbesartan, olmesartan, telmisartan, or any combination thereof.

In other embodiments, for treating cancer in humans, certain compositions disclosed herein can include an agent that inhibits or blocks beta receptor (BRBs) binding or activity, e.g., wherein the BRB in the composition is at a concentration sufficient to provide a dose of at least about 10 mg, at least about 20 mg, at least about 30 mg, at least about 40 mg, at least about 50 mg, at least about 100 mg, at least about 200 mg, at least about 300 mg, at least about 400 mg, at least about 500 mg, at least about 600 mg, at least about 700 mg, at least about 800 mg, including any range between these values, or less than about 400 mg, less than about 300 mg, less than about 200 mg, or less than about 100 mg, including any range between these values. In certain embodiments, the agent that inhibits or blocks beta receptor (BRBs) binding or activity in the composition is at a concentration sufficient to provide a dose of more than about 600 mg, more than about 750 mg, more than about 900 mg, more than about 1050 mg, or more than about 1200 mg, including any range in between these values, or any combinations thereof. Consistent with these embodiments, the BRB can be, but is not limited to, acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, IC1-118,551, SR 59230A, or any combination thereof.

Embodiments disclosed herein concern compositions, methods and uses for inducing immune responses against cancer antigens related thereto in a subject, individually or simultaneously in order to treat cancer in the subject. In accordance with these embodiments, cancer antigens can be generated and used in immunogenic and/or vaccine compositions in combinations as disclosed herein to treat cancer in a subject. In certain embodiments, compositions disclosed here are targeted to certain organs or administered intravenously or systemically by oral or other administration, depending on the tumor type, size, stage of cancer and location and specifics pertaining to the subject.

In certain embodiments, the cancer vaccine can be administered by a subcutaneous or intramuscular route, in conjunction with a suitable vaccine adjuvant (e.g. liposome-TLR adjuvant). In accordance with these embodiments, an exemplary immunization schedule can include oral administration of an ARB and/or a BRB agent. In one embodiment, the dose of the ARB agent could be much higher than the typically prescribed dose (e.g. 5× to 10× the oral dose of losartan) in order to modify the TME enhancing an anti-cancer response in a subject in need thereof. It is also understood that the BRB drugs also modify the TME to enhance tumor immunity and can be administered at a higher dose as necessary. In accordance with these embodiments, administration by these routes can induce a systemic immune response capable of targeting tumors in various locations (e.g. lung, skin, brain, bone, muscle, organs such as liver, kidney, stomach, etc.). In other embodiments, co-administration of an ARB and/or BRB agent modifies the TME making the environment permissive for T cell and antibody responses elicited by the CSC vaccine in order to attach and destroy the tumor or affect immune reactions.

In yet other embodiments, a cancer vaccine can be co-administered with a vaccine adjuvant alone or in combination with an ARB and a BRB agent. In accordance with these embodiments, these agents are capable of having an enhanced effect of modifying the TME in order to treat a subject having cancer such as enhancing T cell activation, reducing tumor size, inhibiting or reducing metastasis or inhibiting or reducing angiogenesis of the tumor, or other side effect of cancer. It is contemplated that these agents can be co-administered to a subject either subcutaneously or intramuscularly depending on the targeted condition to be treated in a subject. Alternatively, in other embodiments, a cancer vaccine in combination with an adjuvant can be administered before, during or after administering an ARB and a BRB agent to a subject in need thereof.

In any one of the embodiments disclosed herein, a subject can be a human or other mammal such as a companion animal (dog, cat), livestock or other farm animal (e.g. horse, cow, sheep, pig), or other animal species either captive or wild.

In certain embodiments, compositions disclosed herein can be used to treat a subject diagnosed with cancer, having a tumor, at risk of spread of an existing cancer (e.g. metastasis) or at risk of developing cancer (e.g. breast cancer or colon cancer). In accordance with these embodiments, a cancer antigen can be derived from one or more tumor(s) of the intended patient suffering from cancer or of a patient that is matched immunologically to the intended patient or from an unrelated/unmatched subject in order to treat the intended patient. It is contemplated that one or more cancer antigen preparations can be formulated from a single tumor or multiple tumors from a donor or the patient having cancer. In accordance with these embodiments, the cancer antigen preparation or lysate can then be delivered to a subject in need of such a treatment, alone and followed by a composition containing a first agent that inhibits or blocks activity and/or binding of an angiotensin II receptor (ARBs); and a second agent that inhibits or blocks beta receptor (BRBs) binding or activity; or the cancer antigen can be combined with a composition containing a first agent that inhibits or blocks activity and/or binding of an angiotensin II receptor (ARBs); and a second agent that inhibits or blocks beta receptor (BRBs) binding or activity and the combined composition delivered to the subject.

Yet other embodiments relate to compositions, methods and uses for treating cancer or a condition associated with cancer. In accordance with these embodiments, methods disclosed herein include methods of treating cancer or a condition associated with cancer in a subject comprising administering a therapeutically effective dose of the pharmaceutical composition disclosed herein. In accordance with these embodiments, pharmaceutical compositions disclosed herein can reduce tumor expansion or metastasis or reduce tumor size in the subject. In certain embodiments, the size of tumor is decreased by 10%, 20%, 30%, 40%, or 50%. In other embodiments, compositions disclosed herein can be used in combination with other known cancer treatments such as surgical removal of tumors, bone marrow transplantation, chemotherapeutic agents, radiation treatment and any other known cancer treatment.

In other embodiments, compositions disclosed herein can be used to induce an immune response in a subject that is immunocompromised, diagnosed with an infection or having reduced or a poor immune response to a condition. In accordance with these embodiments, a composition of use for such conditions can include but are not limited to, a pharmaceutically effective amount of a first agent that inhibits or blocks activity and/or binding of an angiotensin II receptor (ARBs) and a second agent that inhibits or blocks beta receptor (BRBs) binding or activity. In certain embodiment, a pharmaceutically effective amount of a first agent that inhibits or blocks activity and/or binding of an angiotensin II receptor (ARBs) and a second agent that inhibits or blocks beta receptor (BRBs) binding or activity can be used to prime a subject prior to other treatments for their condition or as a follow-up treatment after a more standard treatment of the patient having the condition. In accordance with these embodiments, certain enhanced effects include induction of two types of T cells, CD4+ and CDS+ T cells, to initiate and/or enhance cell mediated immunity and humoral immunity in a subject. For example, compositions disclosed herein can be used to enhance cell-mediated immunity and humoral immunity by upregulating CD4 and CD8 T cells in a subject in need of such enhancement. In certain embodiments disclosed herein, compositions can modulate a subject's immune response, reduce or prevent tumor angiogenesis and/or reduce or block cancer stem cell expansion/growth.

It is currently understood that T cells participate in and initiate cell-mediated or humoral immunity. A T cell, or T lymphocyte, is a type of lymphocyte (a subtype of white blood cell). T cells can be distinguished from other lymphocytes, such as B cells and natural killer cells, by the presence of an identifying T-cell receptor on the cell surface. T cells are named based on their origin as they mature in the thymus from thymocytes (although some also mature in the tonsils). Subsets of T cells each have a distinct function with a majority of human T cells rearrange their alpha and beta chains on the cell receptor and are termed alpha beta T cells (αβ T cells) and are part of the adaptive immune system. Specialized gamma delta T cells, (a small minority of T cells in the human body, more frequent in ruminants), have invariant T-cell receptors with limited diversity that can effectively present antigens to other T cells and are considered to be part of the innate immune system. T helper cells (Th cells) are a type of T cell that plays an important role in the immune system, particularly in the adaptive immune system. T cells can be stimulated to release various cytokines. T cells assist in regulation of immune responses by both suppression and stimulation depending on the circumstances. Th cells are essential in B cell antibody class switching, in the activation and growth of cytotoxic T cells, and in maximizing bactericidal activity of phagocytes such as macrophages.

Mature Th cells express the surface protein CD4 and are referred to as CD4+ T cells. Such CD4+ T cells are generally treated as having a pre-defined role as helper T cells within the immune system. For example, when an antigen-presenting cell expresses an antigen on MHC class II, a CD4+ cell will aid those cells through a combination of cell to cell interactions. CD4 T cells recognize antigenic peptides bound to MHC class II molecules. Upon interaction with a MHC class II molecule, the CD4 cells secret factors such as cytokines which can activate B cells, cytotoxic T cells, macrophages, and other cells that participate in an immune response. Helper T cells or CD4+ cells can be further divided into two functionally distinct subsets. TH1 phenotype and TH2 phenotypes which differ in their cytokine and effector function. Activated TH1 cells enhance cellular immunity and are important for responding to intracellular infections. In other methods, an enhanced immune response can include an enhanced TH1 immune response and/or an enhanced TH2 immune response. In certain embodiments, an enhanced TH1 immune response can include an increase in IgG2a production which can further require a TH1 adjuvant which is known in the art.

A cytotoxic T cell (also referred to as TC, cytotoxic T lymphocyte, CTL, T-killer cell, cytolytic T cell, CD8+ T-cell or killer T cell) is a T lymphocyte (a type of white blood cell) that has been demonstrated to attach and destroy cancer cells, cells that are infected (particularly with viruses), or cells that are damaged in other ways. Most cytotoxic T cells express T-cell receptors (TCRs) that can recognize a specific antigen. An antigen is a molecule capable of stimulating an immune response, and is often produced by cancer cells or viruses. Antigens inside a cell are bound to class I MHC molecules, and brought to the surface of the cell by the class I MHC molecule, where they can be recognized by the T cell. If the TCR is specific for that antigen, it hinds to the complex of the class I MHC molecule and the antigen, and the T cell destroys the cell.

In order for the TCR to bind to the class I MHC molecule, TCRs must contain a glycoprotein called CD8 on their surface, which binds to the constant portion of the class I MHC molecule. These particular T cells are referred to as CD8+ T cells. Affinity between CD8 and the MHC molecule keeps the TC cell and the target cell bound closely together during antigen-specific activation. CD8+ T cells are recognized as TC cells once they become activated and are generally classified as having a pre-defined cytotoxic role within the immune system, However, CD8+ T cells also have the ability to produce cytokines. In certain embodiments, it is contemplated that compositions disclosed herein can be used to specifically manipulate expression of CD4 and/or CD8 T-cells in a treated subject in order to enhance a more specific immune response in the subject, as needed and determined by a health professional.

In certain embodiments disclosed herein, compositions can be present in one or more containers or vials for delivery in a kit or for individual distribution, e.g., single use or multiuse containers or vials. In some embodiments, kits may contain a single combination ARB/BRB tablet in one vial and a cancer vaccine in a separate vial for packaging and delivery. Any composition disclosed herein is contemplated to be part of one or more kits. In various embodiments, a kit or composition can include material for a single administration (e.g. vaccination), or can include material for multiple immunizations (e.g., a “multidose” kit). The inclusion of a preservative can be preferred in multiple dose arrangements. In certain embodiments, a vaccine or an immunogenic composition disclosed herein having an ARB, a BRB and optionally, CSC antigens can be administered to a subject at about 0.1 ml to about 5 ml per dose, about 0.2 ml to about 2 ml per dose, about 0.5 ml to about 2 ml per dose, or about I ml to 2 ml per dose. hi certain embodiments, human vaccines can be administered in a dosage volume of about 0.5 ml, although a reduced volume (e.g. about 0.25 ml) may be administered to children.

In some embodiments, compositions disclosed herein can be provided to a subject on a daily, twice daily, or more, or every other day, a bi-weekly, weekly, hi-monthly, monthly, every other month or other appropriate dosing regimen depending on circumstances or condition of a subject. In certain embodiments, a subject can be treated on a daily basis for a period of time and then a less frequent administration schedule.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regime may be adjusted to provide the optimum therapeutic response.

In one embodiment, the compound (e.g., pharmaceutical chemical, small molecule, compound, protein, peptide etc. of the embodiments) may be administered in a convenient manner, for example, subcutaneous, intravenous, by oral administration, inhalation, intradermal, transdermal application, intradermal, intravenous, intravaginal application or topical application, transdermal (e.g. placement in the ear), intranasal or rectal administration. Depending on the route of administration, the active compound may be contained in a protective buffer (e.g., pharmaceutically acceptable excipient). In one embodiment, a composition may be orally administered. In another embodiment, the composition may be administered intravenously. In one embodiment, the composition may be administered intranasally, such as inhalation. In yet another embodiment, the composition may be administered intradermally using a needle-free system or other intradermal administration system.

A composition may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation The active agent may also be administered parenterally, or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms or other stabilizing formulation.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used. In all cases, the composition can be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g., a compound that induces an immune response to one or more dengue virus serotypes) in the required amount in an appropriate solvent with one or a combination of ingredients enumerated above, as required, followed by filtered sterilization.

Upon formulation, solutions will be administered in a manner compatible with the dosage formulation and in such amount as is therapeutically effective. The formulations are easily administered in a variety of dosage forms, such as the type of injectable solutions described above. It is contemplated that compositions are especially suitable for intramuscular, subcutaneous, intradermal, intranasal and intraperitoneal administration.

The active therapeutic agents may be formulated within a mixture of predetermined ratios for example ARBs and BRBs in a pre-determined amount relative to one another. Single dose or multiple doses can also be administered on an appropriate schedule for a given situation (e.g., treating cancer in a subject). In certain embodiments, formulations can include a tablet form of one or more ARBs in combination with one or more BRBs as disclosed in the concentration ranges described herein. In some embodiments, a single dose of ARB agents and/or BRB agents can be 2 times, 3 times, 4 times, 5 times, or 10 or 20 times more than normally prescribed for other conditions or currently on the market. Alternatively, ratios of ARBs to BRBs can be in a 1:1, 2:1, 1:2, 3:1, 1:3, 4:1, 1:4 up to a 20:1 or 1:20 ratio or as determined by a health professional. In certain embodiments, combinations compositions of ARBs and BRBs can be in the form of a rapidly or slowly dissolving pill to be taken by mouth or delivered by any method known in the art or in the form of a strip (e.g. dissolvable strip) for placement on the skin, cheek, under the tongue or other appropriate placement. In some embodiments, one or more doses of a composition disclosed herein may be introduced to a subject intravenously. In certain methods for administration of compositions disclosed herein administration can occur by an initial higher dose composition followed by maintenance doses; periodic administration such as weekly, biweekly or monthly or other regimen. Deliver of ARB/BRB combination therapies may be administered directly to a tumor site or systemically or both. For example, a high dose composition may be delivered directly to an affected region followed by maintenance closes or the like.

In another embodiment, nasal solutions or sprays, aerosols or inhalants may be used to deliver the compound of interest. Additional formulations that are suitable for other modes of administration include suppositories and pessaries

Certain formulations can include excipients, for example, pharmaceutical grades of mannitol, lactose, starch, magnesium stearate, sodium saccharine, cellulose, magnesium carbonate and the like.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

In certain embodiments, compositions, agents or compounds disclosed herein can be part of a kit of use to treat a subject or a condition (e.g. cancer or immune deficient condition). Kits disclosed herein can include an active agent and at least one container for storing the active agent. In certain embodiments, kits can include a therapeutic composition of a first agent of an ARB and second agent of a BRB either as a single dose agent or provided separately.

Embodiments of the present invention is further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

### EXAMPLES

### Example 1

### Enhancement of Cancer Vaccine Efficacy by Co-Administration of ARB Plus BRB

In one exemplary method, mice were treated with an ARB and a BRB alone or in combination with a cancer vaccine. Referring to FIG. 1, C57B1/6 mice (n=5 per group) with established s.c PyMT tumors (day 7 after tumor injection) were treated by vaccination with a cancer vaccine (Vax), with or without concurrent treatment with losartan (10 mg/kg, once daily, i.p.) or losartan plus propanolol (10 mg/kg once daily, i.p.). The cancer vaccines were prepared by changing tissue culture conditions for a tumor cell line from plastic adherence in complete medium to non-adherent conditions and serum-free medium, which causes the cells to clump and form “spheroids”. The tumor vaccines were prepared by lysing these spheroid cultured cells (CSC antigen source) and mixing with a liposomal adjuvant, and vaccinating the mice once weekly during the study. The combination treatment with vaccine and losartan and propanolol slowed tumor growth much more effectively than vaccination plus losartan alone.

### Example 2

### Combined Treatment with Angiotensin-Receptor Blocker and Beta-Receptor Blocker Controls Tumor Growth More Effective than Either Alone

Referring to FIG. 2, C57B1/6 mice (n=5 per group) with established s.c. PyMT tumors (day 7 after tumor injection) were treated with losartan alone 10 mg/kg, once daily, i.p.) with propranolol (10 mg/kg, i.p., once daily), or with losartan plus propanolol (10 mg/kg once daily, i.p.). The combination treatment with an ARB (e.g. losartan) and a beta blocker (e.g. propranolol) slowed tumor growth much more effectively than either drug alone.

Referring to FIG. 3, C57B1/6 mice (n=5 per group) with established s.c PyMT tumors (day 7 after tumor cell injection) were treated with losartan alone (10 mg/kg, once daily, propanolol alone (10 mg/kg, daily, i.p.) or carvedilol alone (10 mg/kg, daily, i.p.) or losartan plus propanolol (10 mg/kg once daily, i.p.) or losartan plus carvedilol (10 mg/kg once daily, i.p.). At the completion of the study, the mice were euthanized and tumor tissues collected for analysis of immune cells by flow cytometry. Infiltrated immune cells in tumor tissues were identified by gating on CD45⁻ cells (tumor cells were CD45 negative), and also identified by staining for specific T cell markers or markers for macrophages or monocytes.

A combination treatment with an ARB and a beta blocker produced a much stronger T cell infiltrate in tumor tissues (top right panel) than treatment with either drug alone. In particular, treatment with losartan and carvedilol produced an increase in intra-tumoral CD4 T cells, whereas treatment with losartan plus propanolol produced an increase in intra-tumoral CD8⁻ T cells. Notably, none of the drugs administered alone produced these effects. Also, the combination treatments reduced the number of tumor macrophages in tumor tissues, indicating that the drugs, alone or in combination, depleted tumor macrophages as predicted by the in vitro assays.

Referring to exemplary FIG. 4, in certain methods the effects of agents on macrophage infiltration were examined by observing effect of agents on monocyte migration. In one method, monocyte migration inhibitory activity was evaluated in the presence of an ARB (e.g. losartan) and a BRB agent (e.g. propranolol). In these methods, losartan and propranolol were evaluated at relevant serum concentrations (for drug levels achieved by oral dosing) for their ability to block migration of monocytes in vitro. These examples were performed on monocytes from healthy dogs and it was observed that both agents individually demonstrated significant inhibitory activity compared to positive and negative controls in these studies (See for example, FIG. 4).

In another exemplary method, antitumor activity of agents in combination with and without tumor vaccine was analyzed. In these methods, a combination of an ARB and a BRB agent with and without an immunogenic agent (e.g. tumor vaccine) were evaluated for treatment of tumors (e.g. brain tumors). Mice (n=5 per group) with established GL261 brain tumors were treated (e.g. daily) with losartan and propranolol and treated (e.g. once weekly) with an autologous tumor lysate vaccine, alone or in combination. Survival times were significantly longer in animals receiving the combination vaccine and losartan/propranolol treatment, compared to groups receiving the vaccine alone or losartan/propranolol alone (See for example FIG. 5).

In another exemplary method, agents alone and in combination were tested for induction of CD8 T cell as an indicator of immune induction and TIME suppression. In this method, an ARB/BRB combination treatment of an animal model was examined compared to each agent alone. It was demonstrated that a combination of an ARB/BRB compared to each agent alone (e.g. losartan and propranolol) increases CD8 T cell infiltration into tumors. In these methods, tumor infiltrating CD8 T cells were quantified by flow cytometry in PyMT tumor tissues after 15 days of treatment. In tumor tissues of animals treated with the combination of losartan and propranolol, there was a significant increase in infiltrating CD8 T cells, consistent with modulation of the immune suppressive TME (See for example, FIG. 6).

In another exemplary method, agents alone and in combination were tested for induction of CD4 T cell as an indicator of immune induction and TME suppression. In this method. an ARB/BRB combination treatment of an animal model was examined compared to each agent alone. It was demonstrated that a combination of an ARB/BRB compared to each agent alone (e.g. losartan and propranolol) increases CD4 T cell infiltration into tumors. In these methods, tumor infiltrating CD4 T cells were quantified by flow cytometry in PyMT tumor tissues after 15 days of treatment. In tumor tissues of animals treated with the combination of losartan and propranolol, there was an increase in infiltrating CD4 T cells, consistent with modulation of the immune suppressive TME (See for example, FIG. 7).

### Example 3

### Treatment with Angiotensin-Receptor Blocker and Beta-Receptor Blocker in Combination with Tumor Vaccine Induces Tumor Regression in Spontaneous Dog Model of Brain Cancer

In one example, images of effects of an ARB/BRB and tumor vaccine combination were observed on an exemplary tumor (e.g brain tumor) in a pet dog with spontaneous brain cancer for tumor regression/shrinkage. In this example, a tumor response (shrinkage) in the dog to treatment with ARB/BRB combination with tumor vaccine was observed. A canine with malignant glioma (FIG. 8, left panel, pre-treatment) was treated with oral dosing of an ARB/BRB (e.g. losartan and propranolol), in combination with an allogeneic cancer vaccine. No additional treatment was administered to the canine during this experiment. It was observed that after a time, significant tumor regression occurred as demonstrated in the image of the brain on day 30 post-treatment in the MRI images (FIG. 8, right panel, post treatment). The combination therapy can be used for brain tumors and other tumors to reduce tumor size and potentially eliminate the tumor alone or in combination with other anti-cancer therapies and treatments. Given the size, immune and physiological similarities between dogs and humans, and the relevance of the dog spontaneous brain cancer model to human brain cancer, these results provide strong evidence of “reduction to practice” of a novel cancer immunotherapy model relevant to treating brain cancer and other cancers.

### Example 4

### Preparation of CSCs and CSC Antigens

In one exemplary method, cancer vaccines can be prepared in vitro in a manner that increases their immunogenicity. In certain exemplary methods for preparing cancer antigens, tumor cell lines are placed in cell culture conditions designed to increase their immunogenicity and upregulate expression of cancer stem cell antigens. Under standard tissue culture conditions, cancer cells adhere to plastic and are grown in tissue culture medium containing fetal bovine serum (FBS). However, when tumor cells are grown in the absence of FBS and not allowed to adhere to plastic (e.g., non-adherent), cell lysates prepared from these tumor cells become more inherently immunogenic (e.g., stimulate innate immune responses such as NK and DC responses) and also upregulate expression of cancer stem cell antigens.

Cancer vaccines can be produced from a variety of different tumor cells, including primary tumor cells and tumor cell lines. Primary tumor cells are obtained directly from a tumor from an animal having a tumor and these populations can be purified by any of several different techniques known in the art, including flow cytometric cell sorting. Tumor cell lines can be early passage tumor cell lines, or they can be immortalized tumor cell lines. Tumor cells can be derived from any type of tumor, including but not limited to, breast cancer, lung cancer, prostate cancer, colorectal cancer (e.g., colon carcinoma), brain cancer, esophageal cancer, stomach cancer, bladder cancer, pancreatic cancer, cervical cancer, head and neck cancer, ovarian cancer, melanoma, leukemia, myeloma, lymphoma, glioma, Non-Hodgkin's lymphoma, leukemia, multiple myeloma and multidrug resistant cancer.

In other exemplary methods, tumor cells lines and CSCs can be grown in serum-free medium, under non-adherent conditions, introducing one or more growth factors, e.g., one or more growth factors selected from: an epidermal growth factor (EGF), basic fibroblast growth factor (FGF), insulin, insulin-like growth factor (IGF), Notch ligand (e.g., frizzled), Wnt ligand, or certain chemokines (e.g., IL-8) and growth factors (e.g., G-CSF). Typical growth factor or chemokine concentrations used to grow the tumor cells can include 1 ng/ml to 100 ng/ml. In certain methods, the growth factor supplementary tumor cell growth is EGF and/or bFGF. Cells can be cultured for about 1 day to 3 months or between 7 and 40 days (e.g. 30 days) before the cells are harvested for preparation of the stem cell antigens.

In other exemplary methods, tumor cells lines and CSCs can be cultured in serum-free basal medium supplemented with insulin-selenium-transferrin mixture (B27 supplement, prepared by manufacturer, and used at between 10% and 500% with defined cell culture medium (either DME or DME-F12 medium) supplemented with at minimum EGF (e.g. 10 ng/ml) and bFGF (e.g. 10 ng/ml). In certain embodiments, the one or more growth factors are selected from: an epidermal growth factor (EGF), fibroblast growth factor (FGF), G-CSF, Notch ligand, and Wnt ligand. In certain embodiments, the growth factor(s) include bFGF and EGF. In certain embodiments, growth factors can include about 10 ng/ml basic fibroblast growth factor (bFGF) and about 10 ng/ml epidermal growth factor (EGF). In certain methods, cells are cultured on non-stick, Teflon-coated tissue culture plates.

In other exemplary methods, cells cultured under stem cell enrichment conditions can be lysed by any method known in the art such as freeze-thaw and sonication to release and isolate cancer antigens into solution. Proteins and cell membrane materials released from the lysed cancer cultures can be included in a cancer vaccine alone or in combination with other agents. In one illustrative method of preparing antigens for the cancer vaccine, the cancer can be subjected to 3 freeze-thaw cycles to lyse the cells and release antigens into solution. Antigens isolated from these procedures can be incorporated into the cancer vaccine. In certain embodiments, an antigen concentration of about 50 to 100 μg protein per vaccine can be used as determined by for example, BCA assay. In certain methods, this antigen concentration reflects the amount of total protein in the cancer cell lysate.

### Example 5

### Effect of Cell Culture Conditions on Tumor Cell Phenotype

The ability of the cell culture conditions of the present invention to induce changes in the tumor cell phenotype, including expression of cancer stem cell antigens, was demonstrated in several cell lines. CT26 colon carcinoma cells were grown under stem cell enrichment conditions (“CSC cult”) for generation of tumor cells that upregulate expression of stem cell markers, compared to tumor cells cultured under conventional tissue culture conditions (“parent”). CSC culture conditions included growth in serum-free medium supplemented with 1*B-27 mix (Gibco® B27® Supplements available from Gibco, ThermoFisher Scientific), 5 ng/ml bFGF and 5 ng/ml EGF and 1 Glutamax, under non-adherent conditions. Parent conditions included growth in medium (MEMS with 1% non-essential amino acids and penicillin and streptomycin) containing 10% fetal bovine serum, under adherent conditions.

The expression of CSC markers by tumor cells grown under these two different cell culture conditions was determined by flow cytometry analysis using antibodies specific for the cell surface markers, CD29, CD34, CD44, CD90.2, CD117, Sca1, CD133 and Oct3/4, The cells grown under CSC cult conditions showed substantially increased expression of stem cell markers as compared to the cells grown under parent conditions (data not shown). See, e.g., US 2018/0085445.

Cancer vaccines were prepared from tumor cells grown on plastic and with FBS and from tumor cells grown under enhanced immunogenicity and CSC conditions, and subjected to freeze-thaw and sonication to produce cell lysates, which were used as antigens in a CSC vaccine and a conventional tumor vaccine, respectively.

### Example 6

### Effect of Cancer Vaccine in Animal Models of Cancer

Referring to FIG. 9, mice (n=5 per group) with established subcutaneous CT26 tumors were immunized with the enhanced immunogenicity, cancer vaccine (Spheroid VAX) or with a conventional tumor vaccine (Tumor Vax), prepared using lysates from tumor cells grown under conventional adherent conditions with tissue culture medium containing fetal bovine serum, or were unvaccinated (Control). The Spheroid vaccine consisted of 50 μg tumor lysate antigen (from Spheroid cultures) mixed with 200 μl CLDC adjuvant plus 250 mg/ml of RS102895 (or other vaccine enhancing agent). The conventional tumor vaccine included 50 μg tumor antigen (from conventional tumor cultures) mixed with 200 μL CLDC adjuvant plus 250 mg/ml of RS102895 (or other vaccine enhancing agent). Vaccinations were administered SC and repeated weekly. Tumor dimensions were determined at 3-day intervals. As illustrated in FIG. 9, immunization with the CSC vaccine (Spheroid Vax; triangle) led to significant inhibition of tumor growth, compared to animals immunized with a conventional tumor vaccine (Tumor vax: square) or not vaccinated (Control, circle).

Referring to FIG. 10, immune responses in mice vaccinated with CSC vaccines or conventional tumor vaccines were examined. Mice (n=4-5 per group) were vaccinated weekly for 3 immunizations with either standard tumor lysate vaccines (Tumor Vax; squares) or Spheroid lysate vaccines (Spheroid Vax; triangles). Spleen cells from unvaccinated mice and mice immunized with conventional tumor vaccines (Tumor Vax) or enhanced immune vaccines (Spheroid Vax) were then re-stimulated in vitro with cell culture lysates to assess the magnitude of T cell immune responses, as measured by release of interferon-gamma (IFN-gamma). Unvaccinated mice did not generate immune responses (no IFN-gamma release). Mice vaccinated with the enhanced immune vaccine (Spheroid Vax; triangles) mounted much stronger immune responses (IFN-gamma release) than the mice vaccinated with conventional tumor vaccines (Tumor Vax; squares). These data provide evidence that tumor immune responses are increased when animals are vaccinated with CSC as compared to the conventional tumor vaccines. In these exemplary methods, IFN-γ was measured as an indicator of correlated T cell response.

### Example 7

### Culture of Cancer Cells under Spheroid Conditions Generates Cells with Increased Non-Specific Immunogenicity

Referring to FIG. 11, Cancer cells (4T1 mouse mammary cancer cells) were grown in tissue culture under conventional conditions (plastic) or under spheroid conditions (spheroid), which involves serum-free medium and non-adherent conditions designed to increase tumor immunogenicity. After 7 days in culture, the tumor cells in each condition were collected and lysed by freeze-thawing to produce cell lysates. The lysates were then added at a concentration of 50 ug/ml to cultures of mouse spleen cells (either C57B1/6 mice or CD1 mice) and release of a key immune cytokine (IFN-γ) measured 24 h later by ELISA assay. Incubation with lysates from tumor spheroid cultures consistently triggered greater IFN-γ production than lysates from plastic adherent tumor cells, consistent with increased inherent immunogenicity of spheroid grown tumor cells. In certain methods, cancer vaccines prepared using lysates from these tumor cells can be expected to induce a stronger immune response than cancer vaccines prepared from tumor cells grown under conventional conditions (e.g., plastic adherence, FBS containing medium).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

What is claimed:

1. A composition, comprising:
at least a first agent comprising a first agent capable of reducing the activation of, or blocking an angiotensin II receptor (ARB) and at least one of blocking the C-C chemokine receptor 2 (CCR2) and monocyte migration; and
at least a second agent comprising a second agent capable of reducing the activation of, or blocking a beta receptor (beta receptor antagonist, BRB) and blocking monocyte migration.

2. The composition according to claim 1, further comprising a third agent comprising at least one cancer antigen.

3. The composition according to claim 2, wherein the third agent of at least one antigen comprises a lysate of cancer cells grown under non-adherent conditions.

4. The composition according to claim 1, wherein the at least one antigen is obtained from cancer stem cells having undergone at least one freeze-thaw cycle, cancer cells having undergone selective culturing or a cancer cell-containing lysate.

5. The composition according to claim 4, wherein the lysate of the cancer cells is derived in vitro or purified from tumor cell lines derived from one or more of a squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulvar cancer, thyroid cancer, hepatic carcinoma, various types of head and neck cancer, B-cell lymphoma, low grade/follicular non-Hodgkin's lymphoma (NHL), small lymphocytic (SL) NHL, intermediate grade/follicular NHL; intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, chronic lymphocytic leukemia (CLL), acute lymphoblastic leukemia (ALL), Hairy cell leukemia, and chronic and myeloblastic leukemia.

6. The composition according to claim 1, wherein the at least first agent comprises one or more of losartan, azilsartan, candesartan, eprosartan, irbesartan, olmesartan, telmisartan, talsartan, or other ARB agent.

7. The composition according to claim 1, wherein the at least first agent comprises one or more of losartan, irbesartan, telmisartan and talsartan.

8. The composition according to claim 1, wherein the at least second agent comprises one or more of acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, ICI-118,551, SR 59230A, or other BRB agent.

9. The composition according to claim 1, further comprising at least one adjuvant.

10. The composition according to claim 9, wherein the at least one adjuvant comprises at least one of a cationic liposome-DNA complex (CLDC) or a cationic liposome DNA-pIC complex (CLDPC).

11. The composition according to claim 1, wherein the at least first agent comprises losartan and the at least second comprises propranolol.

12. The composition according to claim 1, wherein the composition is formulated for oral single administration.

13. The composition according to claim 1, wherein the concentration of the at least the first or the at least second agent is 2 to 20 times the standard concentration used in other indications currently marketed.

14. A pharmaceutical composition comprising the composition according to claim 1, and a pharmaceutically acceptable excipient.

15. A method for enhancing an immune response in a subject, the method comprising administering a composition according to claim 14 to the subject.

16. The method according to claim 15, wherein the subject has cancer and the method further comprises administering a cancer vaccine to the subject.

17. The method according to claim 15, wherein the subject has cancer and the method further comprises administering one or more checkpoint inhibitors to the subject.

18. The method according to claim 16, wherein the cancer vaccine cell line comprises a cell line derived one or more of a squamous cell carcinoma, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulvar cancer, thyroid cancer, hepatic carcinoma, various types of head and neck cancer, B-cell lymphoma, low grade/follicular non-Hodgkin's lymphoma (NHL), small lymphocytic (SL) NHL, intermediate grade/follicular NHL; intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, chronic lymphocytic leukemia (CLL), acute lymphoblastic leukemia (ALL), Hairy cell leukemia, and chronic myeloblasts leukemia.

19. The method according to claim 15, wherein the subject comprises a human a companion animal, a horse, an agricultural animal or livestock, or other mammal.

20. A kit comprising at least one composition according to claim 1 and at least one container.

21. A composition for modifying a tumor microenvironment (TME), comprising:
an antigen against cancer;
at least a first agent comprising a first agent capable of reducing the activation of, or blocking an angiotensin II receptor (ARB) and the CCR2 receptor; and
at least a second agent comprising a second agent capable of reducing the activation of, or blocking a beta receptor (beta receptor antagonist, BRB).

22. The composition according to claim 21, further comprising at least one adjuvant.

23. The composition according to claim 21, wherein the at least first agent comprises losartan and the at least second agent comprises propranolol.

24. A method for modifying tumor microenvironment (TME) for enhancing an immune response in a subject comprising administering a therapeutically effective amount of the composition according to claim 21, wherein the composition enhances an anti-tumor immune-mediated response in the subject.

25. The method according to claim 24, wherein the method further comprises administering at least one additional T-cell targeted, myeloid cell targeted, adoptive cell transfer, innate immune activating molecule, or metabolic pathway inhibitor immunotherapy or other form of cancer immunotherapy.

26. The method according to claim 25, wherein the at least one additional T-cell targeted immunotherapy comprises one or more checkpoint molecule targeted therapy or adaptive cell therapy.
